(19) United States
(12) Patent Application Publication
(10) Pub. No.: US 2025/0218578 A1
(43) Pub. Date: Jul. 3, 2025

(54) METHODS AND SYSTEMS FOR TO DETERMINE AN APPROPRIATE NEXT DESTINATION FOR TRANSITION OF PATIENT CARE

(71) Applicant: KONINKLIJKE PHILIPS N.V., EINDHOVEN (NL)

(72) Inventor: CLAIRE YUNZHU ZHAO, BOSTON, MA (US)

(21) Appl. No.: 18/833,606

(22) PCT Filed: Feb. 6, 2023
(86) PCT No.: PCT/EP2023/052841

§ 371 Date: Jul. 26, 2024

Related U.S. Application Data
(60) Provisional application No. 63/308,170, filed on Feb. 9, 2022.

(51) Int. Cl. G16H 40/20; G16H 10/60

(52) U.S. Cl. CPC G16H 40/20; G16H 10/60

(57) ABSTRACT

A method (100) for assigning a patient to a next care destination. comprising: (i) receiving (140) a next care destination determination from a care destination determination system (200), wherein the next care destination determination is generated by: receiving (140) information about the patient; analyzing (142), by a trained care destination determination algorithm (264) of the care destination determination system, the received information to generate a next care destination determination; and (ii) transferring (150), based on the received next care destination determination, the patient to the determined next care destination.

### FIELD OF THE DISCLOSURE

The present disclosure is directed generally to methods and systems for assigning a patient to a most appropriate next care destination.

### BACKGROUND

Transition of care requires typically communication and coordination between many different teams and facilities. It also requires knowledge of the patient's condition and progress at the current care setting, prediction of the patient's disease progression, and a determination whether the subsequent care destination is able to support the patient's recovery. As a result, determining an appropriate level of care for a patient's next care setting is a challenge, as well as a vital component of patient outcomes. On one hand, clinicians need to ensure a patient's recovery and long-term prognosis. On the other hand, clinicians must ensure efficient hospital resource utilization so that resources are properly managed and are targeted to the proper patients. The challenge with determining an appropriate level of care is also highlighted by the highly variable admission and discharge rates among different hospitals, even after normalization against confounding factors such as demographics and geographic differences.

Despite these many known challenges in transition of care determinations, there are very few clinical decision support tools that aid in determining the appropriate level of care for a next care destination. While many solutions and machine learning algorithms exist in predicting whether a patient can be discharged, few give predictions on an actual placement of the patient to one of the many various care settings that are available. Furthermore, existing tools only utilize information from the current care setting, with the possible addition of patient history, but they are not informed by any information about next care destinations. As a result, these solutions have limited capacity to determine the appropriateness of a next destination, including how well the patient would be supported in the subsequent care setting given the patient's condition and the resources and capabilities of that subsequent care location.

### SUMMARY OF THE DISCLOSURE

Accordingly, there is a continued need for methods and systems that assign a patient to a most appropriate next care destination. Various embodiments and implementations herein are directed to a trained clinical decision support tool configured to predict how well a patient will be supported in a subsequent care setting, and whether the patient will effectively utilize the care resources at that care setting, in view of the patient's condition and the resources and the capabilities of the subsequent care setting. By providing this prediction, the trained clinical decision support tool helps improve patient recovery and experience, prevents readmissions and resource over-utilization, and overcomes the obstacles in transition points along the care continuum.

Accordingly, various embodiments and implementations herein are directed to a method and system configured to provide information about next care destinations for a patient. A care destination determination system provides a next care destination determination to a clinician. The next care destination determination is generated by the system receiving information about the patient, and then using a trained care destination determination algorithm analyzing the received information to generate a next care destination determination. The clinician can then transfer, based on the received next care destination determination, the patient to the determined next care destination.

Generally, in one aspect, a method for assigning a patient to a next care destination is provided. The method includes: (i) receiving a next care destination determination from a care destination determination system, wherein the next care destination determination is generated by: receiving information about the patient; analyzing, by a trained care destination determination algorithm of the care destination determination system, the received information to generate a next care destination determination; and (ii) transferring, based on the received next care destination determination, the patient to the determined next care destination.

According to an embodiment, the method further includes the step of generating a trained care destination determination algorithm, comprising: (i) receiving a training dataset, comprising treatment data for a plurality of historical patients, the treatment data comprising information for each historical patient about: (1) demographics, diagnosis, and/or treatment, (2) a first care location, and (3) a subsequent care location; (ii) generating a care pathway map, comprising: generating, using the treatment data, a plurality of patient clusters for the historical patients in the training dataset; determining, via process mining, one or more destination process pathways for each of the plurality of patient clusters, based on the first care location(s) and the subsequent care location(s) for the historical patients in the respective patient cluster; (iii) generating a plurality of class labels, comprising repetitions of: assigning a historical patient to at least one of the generated plurality of patient clusters; determining, using a conformance score, how closely the historical patient matches each of the determined destination process pathways associated with the assigned at least one of the generated plurality of patient clusters; normalizing the conformance score to generate a class label; and (iv) training. using the generated plurality of class labels, a care destination determination algorithm to determine a next care destination determination for a new patient, based on one or more of: (1) demographics, diagnosis, and/or treatment of the new patient and (2) a first care location of the patient.

According to an embodiment, the next care destination determination comprises a probability for the next care destination.

According to an embodiment, the next care destination determination comprises two or more possible next care destinations. According to an embodiment, each of the two or more possible next care destinations comprises a probability.

According to an embodiment, the next care destination determination comprises demographic, diagnosis, and/or treatment information about the patient.

According to an embodiment, the next care destination determination is provided via a user interface of the care destination determination system.

According to an embodiment, the next care destination determination is provided via clinical decision support system.

According to a second aspect is a system for assigning a patient to a next care destination. The system includes: (i) a trained care destination determination algorithm; (ii) information about the patient, comprising a current care location for the patient; (iii) a processor configured to analyze, using the trained care destination determination algorithm, the information about the patient to generate a next care destination determination; and (iv) a user interface configured to provide the next care destination determination to a user.

According to an embodiment, the processor is further configured to train a care destination determination algorithm to generate the trained care destination determination algorithm.

According to an embodiment, training the care destination determination algorithm comprises: (i) receiving a training dataset, comprising treatment data for a plurality of historical patients, the treatment data comprising information for each historical patient about: (1) demographics, diagnosis, and/or treatment, (2) a first care location, and (3) a subsequent care location; (ii) generating a care pathway map, comprising: generating, using the treatment data, a plurality of patient clusters for the historical patients in the training dataset; determining, via process mining, one or more destination process pathways for each of the plurality of patient clusters, based on the first care location(s) and the subsequent care location(s) for the historical patients in the respective patient cluster; (iii) generating a plurality of class labels, comprising repetitions of: assigning a historical patient to at least one of the generated plurality of patient clusters; determining, using a conformance score, how closely the historical patient matches each of the determined destination process pathways associated with the assigned at least one of the generated plurality of patient clusters; normalizing the conformance score to generate a class label; and (iv) training, using the generated plurality of class labels, the care destination determination algorithm to determine a next care destination determination for a new patient, based on one or more of: (1) demographics, diagnosis, and/or treatment of the new patient and (2) a first care location of the patient.

According to a third aspect is a method for assigning a patient to a next care destination using a care destination determination system. The method includes: (i) generating a trained care destination determination algorithm, comprising: (a) receiving a training dataset, comprising treatment data for a plurality of historical patients, the treatment data comprising information for each historical patient about: (1) demographics, diagnosis, and/or treatment, (2) a first care location, and (3) a subsequent care location; (b) generating a care pathway map, comprising: generating, using the treatment data, a plurality of patient clusters for the historical patients in the training dataset; determining, via process mining, one or more destination process pathways for each of the plurality of patient clusters, based on the first care location(s) and the subsequent care location(s) for the historical patients in the respective patient cluster; (c) generating a plurality of class labels, comprising repetitions of: assigning a historical patient to at least one of the generated plurality of patient clusters; determining, using a conformance score, how closely the historical patient matches each of the determined destination process pathways associated with the assigned at least one of the generated plurality of patient clusters; and normalizing the conformance score to generate a class label; and (d) training, using the generated plurality of class labels, a care destination determination algorithm to determine a next care destination determination for a new patient, based on one or more of: (1) demographics, diagnosis, and/or treatment of the new patient and (2) a first care location of the patient; (ii) receiving a next care destination determination for a patient, wherein the next care destination determination is generated by: receiving information about the patient; and analyzing, by the trained care destination determination algorithm, the received information to generate a next care destination determination; and (iii) transferring, based on the received next care destination determination, the patient to the determined next care destination.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1A is a flowchart of a method for training a care destination determination algorithm, in accordance with an embodiment.

FIG. 1B is a flowchart of a method for assigning a patient to a next care destination, in accordance with an embodiment.

FIG. 2 is a schematic representation of a care destination determination system, in accordance with an embodiment.

FIG. 3 is a flowchart of a method for training a care destination determination algorithm, in accordance with an embodiment.

FIG. 4A is flowchart of a method for training a care destination determination algorithm, in accordance with an embodiment.

FIG. 4B is flowchart of a method for training a care destination determination algorithm, in accordance with an embodiment.

FIG. 5 is flowchart of a method for training a care destination determination algorithm, in accordance with an embodiment.

FIG. 6 is a flowchart of a method for assigning a patient to a next care destination, in accordance with an embodiment.

FIG. 7 is a schematic representation of a user interface, in accordance with an embodiment.

### DETAILED DESCRIPTION OF EMBODIMENTS

The present disclosure describes various embodiments of a system and method configured to assign a patient to a most appropriate next care destination. More generally, Applicant has recognized and appreciated that it would be beneficial to provide recommendations to clinicians comprising information about appropriate next care destinations in order to improve patient outcomes and maximize facility resources. Accordingly care destination determination system provides a next care destination determination to a clinician. The next care destination determination is generated by the system receiving information about the patient, and then using a trained care destination determination algorithm analyzing the received information to generate a next care destination determination. The clinician can then transfer, based on the received next care destination determination, the patient to the determined next care destination.

According to an embodiment, the methods and systems described or otherwise envisioned herein enable a clinician to better predict or determine whether a patient will efficiently utilize resources and follow a typical care process at a next care destination. The methods and systems can, for example, provide a probability for each available next care destination. Thus, the methods and systems maximize care resource utilization in next care destinations to curate discharge location class labels for each patient in a training set. Then, the systems uses a curated “appropriate discharge location” class label to train a patient placement prediction algorithm, which can be a multi-class, multi-member classifier. The algorithm can be implemented at near real-time and updated frequently or to be triggered at key decision points during patient's stay.

According to an embodiment, the methods and systems described or otherwise envisioned herein provides numerous advantages over the prior art. Providing information about most appropriate next care destination allows for improved patient outcomes and more efficient utilization of facility resources. Both improved patient outcomes and more efficient utilization of facility resources leads to saved lives.

The embodiments and implementations disclosed or otherwise envisioned herein can be utilized with any patient care system, including but not limited to clinical decision support tools, among other systems. For example. one application of the embodiments and implementations herein is to improve analysis systems such as, e.g., the Philips® IntelliSpace® products (manufactured by Koninklijke Philips, N. V.), among many other products. However, the disclosure is not limited to these devices or systems, and thus disclosure and embodiments disclosed herein can encompass any device or system capable of generated and reporting information about next care destinations for a patient.

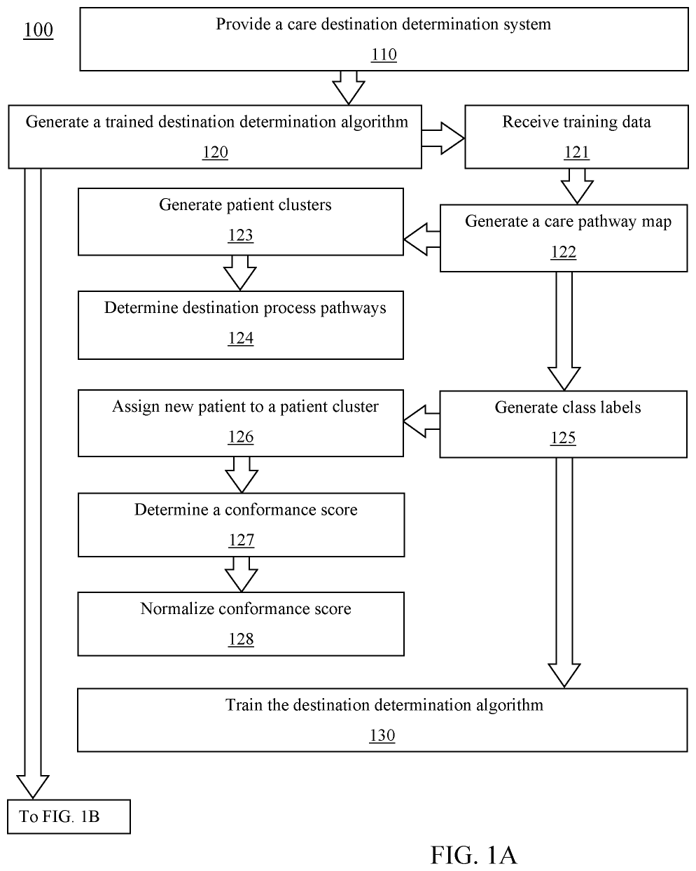

Referring to FIGS. 1A and 1B, in one embodiment is a flowchart of a method 100 for assigning a patient to a next care destination using a care destination determination system. The methods described in connection with the figures are provided as examples only, and shall be understood not to limit the scope of the disclosure. The care destination determination system can be any of the systems described or otherwise envisioned herein. The care destination determination system can be a single system or multiple different systems.

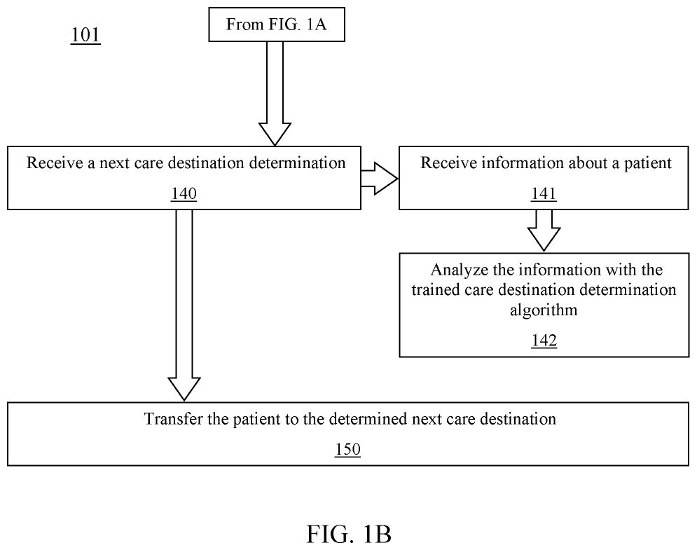

At step 110 of the method, a care destination determination system is provided. Referring to an embodiment of a care destination determination system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, care destination determination system 200 can be any of the systems described or otherwise envisioned herein. Other elements and components of the care destination determination system 200 are disclosed and/or envisioned elsewhere herein.

At step 120 of the method, the care destination determination system generates a trained destination determination algorithm. The trained destination determination algorithm is configured to analyze input data in order to generate an output. According to an embodiment, the output of the trained destination determination algorithm is a recommendation or classification of the patient for a next care destination, a likelihood or probability of a next care destination, or similar recommendation or classification, among other possible output. The trained destination determination algorithm can be any algorithm capable of creating the output, including but not limited to machine learning algorithms, classifiers, and other algorithms. The trained destination determination algorithm is a unique algorithm based on the training data used to train the algorithm.

The trained destination determination algorithm can be generated by many different mechanisms. According to an embodiment, the trained destination determination algorithm is generated according to the method depicted in FIG. 1A, although this example is a non-limiting example of training.

According to this embodiment, at step 121 of the method, the care destination determination system 200 receives, retrieves, or otherwise obtains training data that will be utilized to train the algorithm. The training data can be any data that will be utilized to train the algorithm. For example, the training data can comprise treatment data for a plurality of historical patients, the treatment data comprising information for each historical patient about: (1) demographics, diagnosis, and/or treatment, (2) a first care location, and (3) a subsequent care location. The training data can comprise any other information.

According to an embodiment, the training data comprises clinical data that measures patient physiology—such as physiological measurements of vital signs, lab results, imaging results, and other data—and event logs—such as timestamps of orders of labs, imaging, medication, consultation, and other data—that reflect the procedures the patient received in the current care setting. These will form data set {Dₙ}, or the data from the current care location n, and data set {Dₙ₊₁}, or the data from the next care location n+1.

The training data can be received, retrieved, or otherwise obtained from an electronic medical record database or system 270. The electronic medical records database may be a local or remote database and is in direct and/or indirect communication with the care destination determination system 200. The training data may be stored in and/or received from one or more databases. The database may be a local and/or remote database. For example, the care destination determination system may comprise a database of training data.

According to an embodiment, the care destination determination system may comprise a data pre-processor or similar component or algorithm configured to process the received training data. For example, the data pre-processor analyzes the training data to remove noise, bias, errors, and other potential issues. The data pre-processor may also analyze the input data to remove low-quality data. Many other forms of data pre-processing or data point identification and/or extraction are possible.

At step 122 of the method, the care destination determination system generates a care pathway map. The care pathway map comprises information about possible current care locations, possible subsequent care locations, and associations between current care locations and subsequent care locations, among other information. Once generated, the care pathway map can be utilized immediately in subsequent downstream steps of the method, or may be stored in local and/or remote memory for future use of the information.

In order generate the care pathway map, at step 123 of the method the system utilizes the treatment data and the historical patients in the training data to generate a plurality of patient clusters. The patient clusters {Cⱼ} can be generated using any method for grouping or clustering data. For example, the patient clusters can be generated using sequence clustering, hierarchical clustering, Principal Component Analysis (PCA), Locally Linear Embedding (LLE), and/or any other clustering mechanism. Once generated, the patient clusters can be utilized immediately in subsequent downstream steps of the method, or may be stored in local and/or remote memory for future use of the information.

At step 124 of the method, the system determines one or more destination process pathways for each of the plurality of patient clusters, based on the first care location(s) {n} and the subsequent care location(s) {Lᵢ} for the historical patients in the respective patient cluster. According to an embodiment, for each patient cluster {Cⱼ}, for each care location {Lᵢ}, the system obtains process pathways {P_{i,j,k}} (where i is a next care destination, j is a cluster, and k is a process pathway) from the data from the next care location {Dₙ₊₁}.

Many different methods for determining destination process pathways are possible. For example, the system can utilize process mining techniques such as an inductive miner, a heuristic miner, and many other techniques. According to an embodiment, process mining uses information such as event logs, patient treatment locations, and other information to generate possible patient flow avenues such as possible subsequent treatment locations for patients. For example, a patient cluster may comprise 100patients located within a first location (such as an ICU), and the training data for those patients (such as event logs, timestamps, etc.) may indicate that those 100 patients went to nine (9) different subsequent locations. Process mining may determine not only to which locations those 100 patients in the patient cluster went, but a circumstance indicating why the patient went to the location, frequencies, and other associated information.

According to one possible embodiment, in order to improve the quality and usefulness of the clinical pathways generated, the determined pathways can be curated and/or selected based on clinical knowledge. They can also be curated and/or selected based on quantitative measures without clinical knowledge, such as sequence length, support, verification of by how close an unseen validation data set matches the discovered process pathway {P_{i,j,k}}. In this way, the number of process pathways, k, can be determined.

According to an embodiment, steps 123 and 124 can be repeated many times to tune hyperparameters or obtain new data or use new data preprocessing for the input clinical features and event logs, until satisfactory P_{i,j,k}'s are obtained.

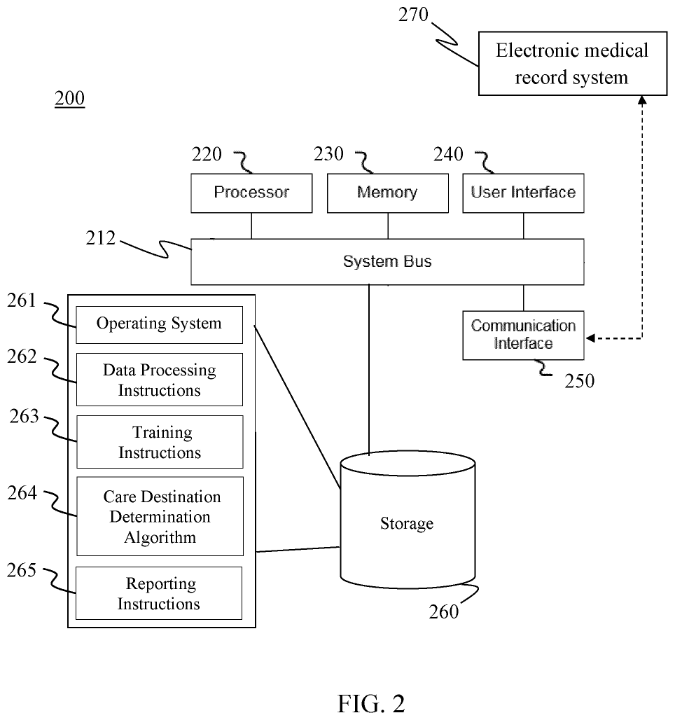

Referring to FIG. 3, in one embodiment, is a flowchart of a method 300 for discovering or determining care pathways using a process mining technique such as the method described above. At step 310, the system receives training data comprising at least clinical features and event logs, although the training data can comprise much more information as well. The system first discovers, determines, or generates a plurality of patient clusters {Cⱼ} using data from the current clinical location {Dₙ} for the historical patients in the training data. For example, at step 320, the system clusters the patients into similar groups using a clustering algorithm as described or otherwise envisioned herein. This results in a plurality of patient clusters {Cⱼ} being output at step 330 of the method.

The system then obtains process pathways {P_{i,j,k}} for each patient cluster from data for the next care location {Dₙ₊₁}. Thus, at step 340, for each cluster of patients {Cⱼ}. for each care destination {Lᵢ}, a process discovery algorithm (such as an inductive miner) determines one or more process pathways. This results in one or more process pathways {P_{i,j,k}} associated with each patient cluster {Cⱼ}.

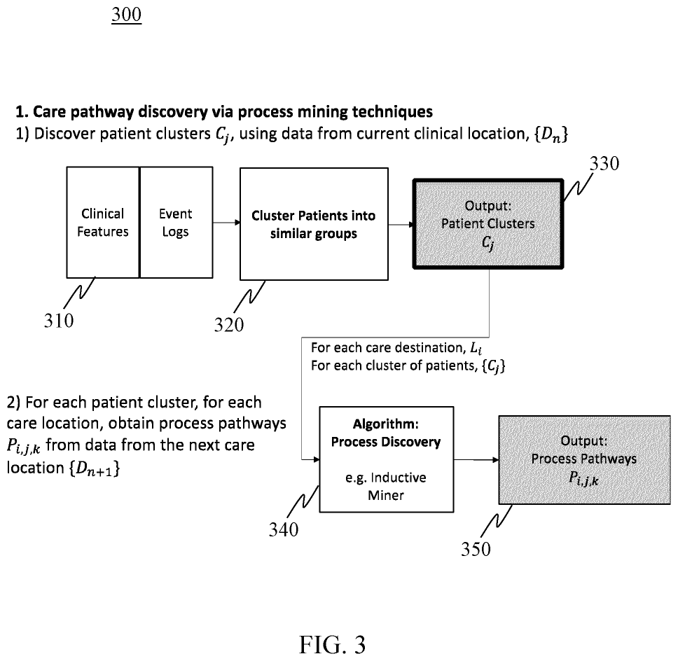

Similarly, referring to FIG. 4A, clusters are assigned using data from the current clinical location {Dₙ} for the historical patients in the training data, the training data 410 comprising at least clinical features and event logs, although the training data can comprise much more information as well. The training data is used to assign patients to a plurality of patient clusters {Cⱼ} at 420, and then at 430 the system obtains process pathways {P_{i,j,k}} for each patient cluster from data for the next care location {Dₙ₊₁}.

At step 125 of the method in FIG. 1, the care destination determination system generates a plurality of class labels using conformance checking. These class labels can be utilized to train the destination determination algorithm. Once generated, the class labels can be utilized immediately in subsequent downstream steps of the method, or may be stored in local and/or remote memory for future use of the information.

In order generate the class labels, at step 126 of the method the system assigns a historical patient from the training dataset, or a new dataset, to at least one of the generated plurality of patient clusters. Thus, according to an embodiment, a patient is assigned to one of the clusters using data from the patient's current care location. According to an embodiment, the cluster assignment is obtained using the smallest distance to a cluster based on distance metrics utilized herein. According to an embodiment, a single patient case can be restricted to be assigned to only one cluster, or can be assigned to two or more clusters. Distance thresholds can be determined empirically, or can be programmed or otherwise determined by a user. For example, to improve predictions, a user may require distances to be smaller.

At step 127 of the method, the system determines how closely the historical patient matches each of the determined destination process pathways associated with the assigned at least one of the generated plurality of patient clusters, based on a conformance score. According to an embodiment, therefore, the system determines how similar the patient's data in the subsequent care setting is to all pathways associated with the clusters to which the patient is assigned, across all possible subsequent care locations. This is the conformance checking. The conformance scores generated indicate the extent to which the particular patient's data matches the discovered process pathways, P_{i,j,k}. Possible techniques include relay, trace alignment, and behavioral alignment, among other methods.

At step 128 of the method, the conformance scores are normalized to generate a class label. According to an embodiment, the conformance scores are normalized to obtain probabilities for the patient to be appropriately placed in each of the possible care locations, which will be used as class labels for the next stage. Thus, according to an embodiment, the output of the conformance checking, i.e. conformance scores, will be normalized against the maximum of the sums across the care locations for a patient, across all patients.

According to an embodiment, steps 126 through 128 are repeated many times each with a new patient until a suitable number of historical patients are utilized, and/or until a suitable number of normalized conformance scores, or class labels, are generated.

Referring to FIG. 4B, in one embodiment, is a method for obtaining placement probabilities using the next care location {Dₙ₊₁}. At step 440, the system generates conformance scores, checking conformance of historical patients in the training dataset against the discovered process pathways, P_{i,j,k}. At step 450, the conformance scores are normalized to generate class labels. The output is thus a probability of a patient p being placed in a destination Lᵢ.

At step 130 of method 100 in FIG. 1A, the care destination determination system 200 trains the destination determination algorithm based on the generated class labels, using data from the current care location. According to an embodiment, the destination determination algorithm can be any algorithm trained or programmed to utilize any or all of the input described or otherwise envisioned herein, and trained or programmed to generate and provide any or all of the output described or otherwise envisioned herein. According to an embodiment, the destination determination algorithm is a trained machine learning algorithm trained to utilize the input and to generate the output as described or otherwise envisioned herein.

According to an embodiment, a multi-label classifier can be deployed to obtain probabilities of placement across all possible destinations, Lᵢ. Alternatively, according to another embodiment, argmax can be taken on class label probabilities to obtain the most likely placement. As such, a multi-class classifier can be obtained for simplification, where single placement is predicted for a patient. Examples of such classifiers include xgboost algorithm, gradient boosting, random forest and other ensemble techniques. According to an embodiment, other deep learning techniques such as neural networks can also be deployed, depending on the complexity of the problem. For multi-class algorithm, the listed algorithm can be deployed in a One versus One (OvO) or One versus All (OvA) scheme. Many other methods are possible.

According to an embodiment, one or more steps of the method of FIG. 1A is conducted or repeated until the destination determination algorithm is sufficiently and/or satisfactorily trained. Determining whether the destination determination algorithm is sufficiently and/or satisfactorily trained can be based on testing of the algorithm, one or more curated or reviewed parameters of the algorithm, or via any other method for determining suitability.

Thus, the output of the method depicted in FIG. 1A is a trained destination determination algorithm and can be utilized and deployed as needed or desired. Once generated, the trained destination determination algorithm can be utilized or deployed immediately, or it may be stored in local and/or remote memory for future use and/or deployment.

The trained destination determination algorithm can then be used to generate next care destination recommendations for patients. A generated next care destination recommendations can be provided to a clinician to facilitate decision-making. The generated next care destination recommendations can be provided to a clinician via a user interface of the care destination determination system 200, or via a user interface of another system in communication with the care destination determination system, such as a hospital or other care setting system.

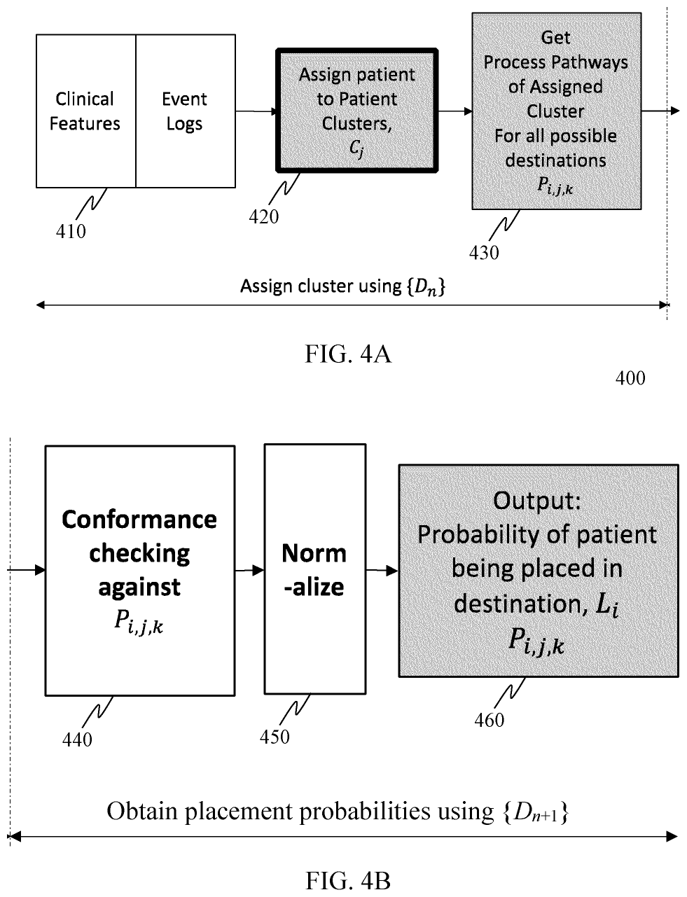

Referring to FIG. 5. in one embodiment. is a method 500 for training the destination determination algorithm, which can be called the Patient Placement Prediction Algorithm. According to an embodiment, the Patient Placement Prediction Algorithm is a multi-label classifier. The Patient Placement Prediction Algorithm receives training input comprising class labels 510, which are the probability of a patient being placed in a destination {Prob_{p,j}}, and clinical features {Dₙ}.

Referring to FIG. 1B, in one embodiment, is a flowchart of a method 101 for using the trained destination determination algorithm to generate and implement a next care destination recommendation for a patient. The method described in conjunction with this figure is provided as an example only, and shall be understood not to limit the scope of the disclosure.

At step 140 of the method, the care destination determination system provides a next care destination recommendation for a patient generated by a trained destination determination algorithm of the system. The generated next care destination recommendations can be provided to a clinician via a user interface of the care destination determination system 200, or via a user interface of another system in communication with the care destination determination system, such as a hospital or other care setting system.

In order to generate the next care destination recommendation by the trained destination determination algorithm, the system receives, retrieves, or otherwise obtains information about a patient. The information is any information required to make a next care destination recommendation, such as demographic, diagnosis, and/or treatment information about the patient, and/or current care location about the patient. The patient information can be received, retrieved, or otherwise obtained from any source, including but not limited to an electronic medical record database or system 270. The source of patient information may be a local or remote database and can be in direct and/or indirect communication with the care destination determination system 200. The patient information may be stored in and/or received from one or more databases. The database may be a local and/or remote database. For example, the care destination determination system may comprise a database of patient information.

At step 142 of the method, the trained care destination determination algorithm of the care destination determination system analyzes the received information to generate a next care destination determination. According to an embodiment, the algorithm uses the received information to predict the probabilities of patient being placed in the available care destinations, using data from the current care location. Based on the probabilities, the physician can decide where the patient's next destination should be.

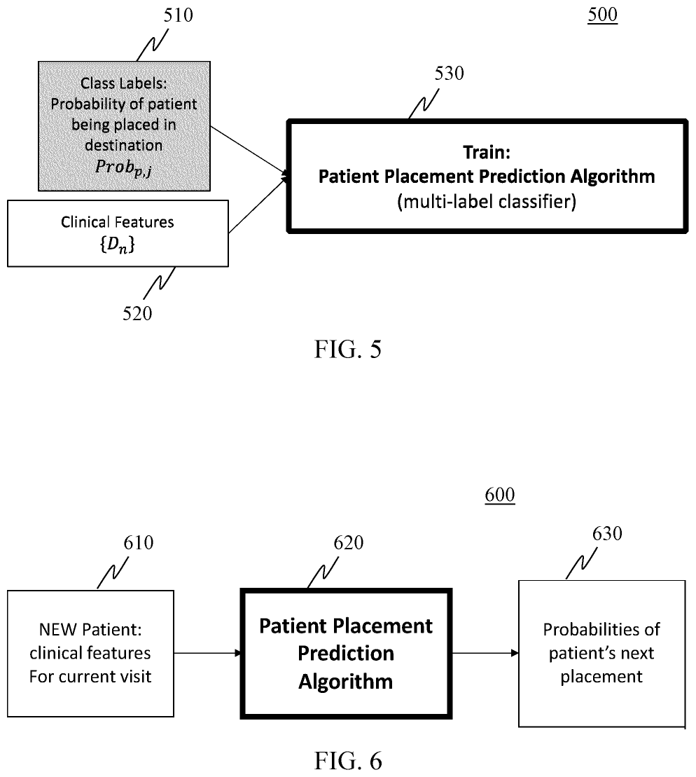

Referring to FIG. 6, in one embodiment, is a method 600 for generating class labels that reflect appropriateness of next care destination placement for patients, using the trained care destination determination algorithm of the care destination determination system. At 610, the system receives information about a new patient, including clinical features for the current visit, such as patient demographics, treatment, and/or diagnosis along with the current care location. At 620 the trained care destination determination algorithm of the care destination determination system analyzes the information from 610, and generates probabilities 630 of the patient's next placement.

The output of the trained care destination determination algorithm of the care destination determination system is utilized by the clinician in their decision-making. Thus, the output can be provided to the clinician using any method for communicating information. According to an embodiment, the output is provided to the clinician via a user interface, such as a user interface of system 200, or another user interface that the clinician utilizes. For example, the output can be provided via a user interface of a clinical decision support tool, among many other user systems and interfaces.

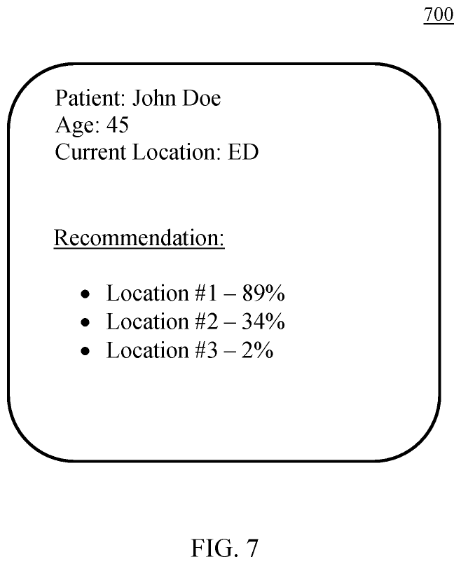

Referring to FIG. 7, in accordance with an embodiment, is a non-limiting example of an output 700 of the trained care destination determination algorithm of the care destination determination system, as provided to a clinician via a user interface of a care system or other communication system. In this example, patient John Doe is 46 and currently located in the Emergency Department of a hospital or similar care setting. The display comprises demographic information about John Doe, including their age (46), among other possible demographic information. Although not shown in this example, the display, output, and/or recommendation may further comprise information about John Doe such as diagnosis information, treatment information, historical treatment or admission information, and/or any other information.

In addition, the display comprises the location output of the trained care destination determination algorithm of the care destination determination system. For example, in FIG. 7, the location output comprises information about three possible next care destinations (Location #1, Location #2, and Location #3), and a probability or recommendation for each of the three possible next care destinations. The next care destination may be any location to which the patient may be transferred, including but not limited to discharge, hospital admission, staying in the same location, long-term care, rehabilitation, and many other possible locations.

Accordingly, at step 150 of the method, the clinician utilizes the received next care destination determination to transfer the patient to the recommended next care destination. Transferring the patient may comprise creating an order to transfer the patient to the next care destination, physically transferring the patient to the next care destination, and/or other steps to move the patient to the next care destination.

Referring to FIG. 7, in one non-limiting embodiment, Location #1 may refer to hospital admission (at 89%). Thus, based on the location output of the trained care destination determination algorithm of the care destination determination system, the clinician admits the patient to the hospital from the Emergency Department.

Accordingly, the care destination determination system utilizes retrospective patient data to train a care destination determination algorithm. The system discovers care pathways via process mining techniques. by which the system uses data including first (i.e., current) care location and generates patient clusters. For each patient cluster, for each possible care location, the system uses process mining techniques to obtain process pathways from the training data for next care location(s). The system then generates class labels via conformance checking. For example, using data from the first (i.e., current) care location, the system can assign a new patient-such as a new patient from training data-to one of the plurality of generated patient clusters. The system can then check to determine how similar the patient's data in the subsequent care setting is to all pathways associated with the clusters to which the patient has been assigned, across all possible subsequent care locations (i.e., conformance checking). The system can then normalize conformance scores to obtain probabilities for the patient for each of the next possible care location, which will be used as class labels for the next stage. The system trains the next care location determination algorithm based generated class labels, using data from the first (i.e., current) care location. Once the algorithm is trained, the algorithm is used for a new patient to predict the probabilities of the patient being placed in next available care destinations, using data from the patient's current care location. Based on the probabilities, the physician can decide where the patient's next care destination will be.

According to an embodiment, the output of the trained care destination determination algorithm of the care destination determination system can further comprise predicted care process pathways in the subsequent settings for the patient, such that the care team in the current care setting can interpret the disposition placement predictions outputted by the algorithm and further evaluate the decision according to their own expertise.

According to an embodiment, individual predictions or recommendations or other output of the trained care destination determination algorithm of the care destination determination system can be aggregated to a population level to improve the prediction of patient flows across care settings, to provide insights to un-anticipated/un-scheduled admissions in the subsequent care setting, and to check whether workflows deviate from a typical process, desired workflow, guidelines, or other predetermined workflow. Thus, the system helps a clinical team with transitions of care, and helps facilitate patient flows in care settings.

According to one non-limiting embodiment, the system may be utilized for patients in an Emergency Department (ED) setting. There are many possible next care locations when patients are discharged from an ED, including the General Ward (GW) or floor, ICU, long-term care facilities, nursing homes, and the patient's own home, among other care locations. This disposition decision is often complicated and sometimes involves communication between the two care settings in the hand-over. For instance, most patients in the ICU come from the ED, and thus the ICU attending must sometimes talk to the ED physician or directly walk down to the ED to see the patient. ICU beds are limited and must be prioritized for the sickest patients. For the ED clinicians, the system can help predict whether the patient will effectively utilize the care level designated in the ICU and determine whether the patient could also possibly be accommodated in less critical settings. In addition, the system can also output predicted care plans in subsequent settings for the patient for the clinical team to further evaluate the disposition decision.

According to another embodiment, similar to the ED example described above, there are many possible discharge locations from the GW or floor, including ICU. transitional care facilities (TCU), long-term care facilities. and nursing homes, and the patient's home, among other care locations. The system can help support the clinical team's placement decisions for next steps in the patient's care. Possible next stage care pathway displays tailored for the patient can provide interpretation of algorithm outputs for the clinicians.

Referring to FIG. 2 is a schematic representation of a care destination determination system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, the electronic medical record system 270 is an electronic medical records database from which the information about a plurality of patients, including demographic, diagnosis, and/or treatment information may be obtained or received. According to an embodiment, the electronic medical record system 270 is an electronic medical records database from which the training data utilized to train the care destination determination algorithm. The training data can be any data that will be utilized to train the algorithm. For example, the training data can comprise treatment data for a plurality of historical patients, the treatment data comprising information for each historical patient about: (1) demographics, diagnosis, and/or treatment, (2) a first care location, and (3) a subsequent care location. The training data can comprise any other information. The electronic medical records database may be a local or remote database and is in direct and/or indirect communication with system 200. Thus, according to an embodiment, the care destination determination system comprises an electronic medical record database or system 270.

According to an embodiment. storage 260 of system 200 may store one or more algorithms, modules, and/or instructions to carry out one or more functions or steps of the methods described or otherwise envisioned herein. For example, the system may comprise, among other instructions or data, data processing instructions 262, training instructions 263, trained care destination determination algorithm 264. and/or reporting instructions 265.

According to an embodiment, data processing instructions 262 direct the system to retrieve and process input data which is used to train the care destination determination algorithm 264. The data processing instructions 262 direct the system to, for example, receive or retrieve input data to be used by the system as needed, such as from electronic medical record system 270 among many other possible sources. As described above, the input data can comprise a wide variety of input types from a wide variety of sources. According to an embodiment, the data processing instructions 262 also direct the system to process the input data to generate training data which is used to train the classifier. This can be accomplished by a variety of embodiments for, for example, feature identification, extraction, and/or processing. The outcome of the processing is a training data set that can be utilized to train the care destination determination algorithm 264.

According to an embodiment, training instructions 263 direct the system to utilize the processed data to train the care destination determination algorithm 264. The care destination determination algorithm can be any algorithm, classifier, or model sufficient to utilize the type of input data provided, and to generate the next care location determination output. Thus, the system comprises a trained care destination determination algorithm 264 configured to generate a recommendation analysis for a patient, as described or otherwise envisioned herein.

According to an embodiment reporting instructions 265 direct the system to generate and provide a user via a user interface information comprising a next care location recommendation. Alternatively, the information may be communicated by wired and/or wireless communication to another device. For example, the system may communicate the information to a mobile phone, computer, laptop, wearable device, and/or any other device configured to allow display and/or other communication of the information.

According to an embodiment. the care destination determination system is configured to process many thousands or millions of datapoints in the input data used to train the care destination determination algorithm, as well as to process and analyze the vast plurality of patient data. For example, generating a functional and skilled trained care destination determination algorithm using an automated process such as feature identification and extraction and subsequent training requires processing of millions of datapoints from input data and the generated features. This can require millions or billions of calculations to generate a novel trained care destination determination algorithm from those millions of datapoints and millions or billions of calculations. As a result, each trained care destination determination algorithm is novel and distinct based on the input data and parameters of the machine learning algorithm, and thus improves the functioning of the care destination determination system. Thus, generating a functional and skilled trained care destination determination algorithm comprises a process with a volume of calculation and analysis that a human brain cannot accomplish in a lifetime, or multiple lifetimes.

In addition, the care destination determination system can be configured to continually receive patient data, perform the analysis, and provide periodic or continual updates via the report provided to a user for the patient. This requires the analysis of thousands or millions of datapoints on a continual basis to optimize the reporting, requiring a volume of calculation and analysis that a human brain cannot accomplish in a lifetime.

By providing an improved next care location recommendation for a patient, this novel care destination determination system has an enormous positive effect on patient management and care compared to prior art systems. As just one example in a clinical setting, by providing a system that can provide improved next care location recommendation for a patient, the system can facilitate and improve treatment, thereby improving patient care, increasing efficiency, and reduce costs.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of.” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for assigning a patient to a next care destination, comprising:
receiving a next care destination determination from a care destination determination system, wherein the next care destination determination is generated by:
receiving information about the patient;
analyzing, by a trained care destination determination algorithm of the care destination determination system, the received information to generate a next care destination determination;
transferring, based on the received next care destination determination, the patient to the determined next care destination.

2. The method of claim 1, further comprising the step of generating a trained care destination determination algorithm, comprising:
receiving a training dataset, comprising treatment data for a plurality of historical patients, the treatment data comprising information for each historical patient about: (i) one or more of demographics, diagnosis, and/or treatment, (ii) a first care location, and (iii) a subsequent care location;
generating a care pathway map, comprising:
generating, using the treatment data, a plurality of patient clusters for the historical patients in the training dataset;
determining, via process mining, one or more destination process pathways for each of the plurality of patient clusters, based on the first care location(s) and the subsequent care location(s) for the historical patients in the respective patient cluster;
generating a plurality of class labels, comprising repetitions of:
assigning a historical patient to at least one of the generated plurality of patient clusters;
determining, using a conformance score, how closely the historical patient matches each of the determined destination process pathways associated with the assigned at least one of the generated plurality of patient clusters;
normalizing the conformance score to generate a class label;
training , using the generated plurality of class labels, a care destination determination algorithm to determine a next care destination determination for a new patient, based on one or more of: (i) one or more of demographics, diagnosis, and/or treatment of the new patient and (ii) a first care location of the patient.

3. The method of claim 1, wherein the next care destination determination comprises a probability for the next care destination.

4. The method of claim 1, wherein the next care destination determination comprises two or more possible next care destinations.

5. The method of claim 4, wherein each of the two or more possible next care destinations comprises a probability.

6. The method of claim 1, wherein the next care destination determination comprises demographic, diagnosis, and/or treatment information about the patient.

7. The method of claim 1, wherein the next care destination determination is provided via a user interface of the care destination determination system.

8. The method of claim 1, wherein the next care destination determination is provided via clinical decision support system.

9. A system for assigning a patient to a next care destination, comprising:
a trained care destination determination algorithm;
information about the patient, comprising a current care location for the patient;
a processor configured to analyze, using the trained care destination determination algorithm, the information about the patient to generate a next care destination determination; and
a user interface configured to provide the next care destination determination to a user.

10. The system of claim 9, wherein the processor is further configured to train a care destination determination algorithm to generate the trained care destination determination algorithm.

11. The system of claim 10, wherein training the care destination determination algorithm comprises:
receiving a training dataset, comprising treatment data for a plurality of historical patients, the treatment data comprising information for each historical patient about: (i) one or more of demographics, diagnosis, or treatment, (ii) a first care location, and (iii) a subsequent care location;
generating a care pathway map, comprising:
generating, using the treatment data, a plurality of patient clusters for the historical patients in the training dataset;
determining, via process mining, one or more destination process pathways for each of the plurality of patient clusters, based on the first care location(s) and the subsequent care location(s) for the historical patients in the respective patient cluster;
generating a plurality of class labels, comprising repetitions of:
assigning a historical patient to at least one of the generated plurality of patient clusters;
determining, using a conformance score, how closely the historical patient matches each of the determined destination process pathways associated with the assigned at least one of the generated plurality of patient clusters;
normalizing the conformance score to generate a class label;
training, using the generated plurality of class labels, the care destination determination algorithm to determine a next care destination determination for a new patient, based on one or more of: (i) one or more of demographics, diagnosis, or treatment of the new patient and (ii) a first care location of the patient.

12. The system of claim 9, wherein the next care destination determination comprises a probability for the next care destination.

13. The system of claim 9, wherein the next care destination determination comprises two or more possible next care destinations.

14. The system of claim 9, wherein the next care destination determination is provided via clinical decision support system.

15. A method for assigning a patient to a next care destination using a care destination determination system, comprising:
generating a trained care destination determination algorithm, comprising:
receiving a training dataset, comprising treatment data for a plurality of historical patients, the treatment data comprising information for each historical patient about: (i) one or more of demographics, diagnosis, or treatment, (ii) a first care location, and a subsequent care location;
generating a care pathway map, comprising:
generating, using the treatment data, a plurality of patient clusters for the historical patients in the training dataset;
determining, via process mining, one or more destination process pathways for each of the plurality of patient clusters, based on the first care location(s) and the subsequent care location(s) for the historical patients in the respective patient cluster;
generating a plurality of class labels, comprising repetitions of:
assigning a historical patient to at least one of the generated plurality of patient clusters;
determining, using a conformance score, how closely the historical patient matches each of the determined destination process pathways associated with the assigned at least one of the generated plurality of patient clusters; and
normalizing the conformance score to generate a class label; and
training, using the generated plurality of class labels, a care destination determination algorithm to determine a next care destination determination for a new patient, based on one or more of: (i) one or more of demographics, diagnosis, or treatment of the new patient and (ii) a first care location of the patient;
receiving a next care destination determination for a patient, wherein the next care destination determination is generated by:
receiving information about the patient;
analyzing, by the trained care destination determination algorithm, the received information to generate a next care destination determination; and
transferring, based on the received next care destination determination, the patient to the determined next care destination.
